(19) United States
(12) Patent Application Publication
(10) Pub. No.: US 2010/0057017 A1
(43) Pub. Date: Mar. 4, 2010

(54) FEEDING SYRINGE ASSEMBLY FOR AN ENDOSCOPIC GASTRONOMY TUBE

(76) Inventors: JOHN PAPPAS, Scottsdale, AZ (US); DeLaine Pappas, Scottsdale, AZ (US)

Correspondence Address: MICHAEL WINFIELD GOLTRY, 4000 N. CENTRAL AVENUE, SUITE 1220, PHOENIX, AZ, 85012, US

(21) Appl. No.: 12/537,673

(22) Filed: Aug. 7, 2009

Related U.S. Application Data
(60) Provisional application No. 61/087,264, filed on Aug. 8, 2008.

(51) Int. Cl. A61M 5/14

(52) U.S. Cl. 604/257

(57) ABSTRACT

A feeding syringe assembly includes a tube coupling a patient in fluid communication with a syringe to convey a formulated food solution to the patient to provide the patient with nutrition, the tube is connected to the patient through an insertion point, a stand, and a syringe support coupling the syringe to the stand holding the syringe at an elevated location with respect to the insertion point of the tube to allow a formulated food solution to gravity convey through the tube to the patient from the syringe.

### CROSS-REFERENCE TO RELATED APPLICATION

This application claims the benefit of U.S. Provisional Patent Application Ser. No. 61/087,264, filed Aug. 8, 2008.

### FIELD OF THE INVENTION

The present invention relates to patient feeding systems and, more particularly, to syringes for endoscopic tubes to convey formulated food into the stomachs of patients.

### BACKGROUND OF THE INVENTION

Patients with various throat or mouth ailments are often fed with a percutaneous endoscopic gastronomy (PEG) tube, which conveys a formulated food solution directly into the patient's stomach. Typically, a battery-powered or electrical pump propels the solution through the tube. However, the performance of battery-powered pumps deteriorates rapidly as the batteries become depleted. Furthermore, in certain situations a patient may require feeding in locations where conventional electrical power is unavailable.

### SUMMARY OF THE INVENTION

Accordingly, there is need in the art for a convenient and reliable system of applying formulated food solutions into a PEG tube that utilizes gravity to force formulated food solutions through a feeding tube.

According to the principle of the invention, feeding syringe assembly includes a tube coupling a patient in fluid communication with a syringe to convey a formulated food solution to a patient to provide the patient with nutrition, the tube connected to the patient at an insertion point, a stand, and a syringe support coupling the syringe to the stand holding the syringe at an elevated location with respect to the insertion point of the tube to allow a formulated food solution to gravity convey through the tube to the patient from the syringe. The syringe support includes a clamp connected to the stand releasably gripping the syringe. The clamp is a C-clamp in a preferred embodiment. The stand includes base and a riser extending substantially vertically upright therefrom to an upper end, and the syringe support is attached to the riser proximate to the upper end of the stand.

### BRIEF DESCRIPTION OF THE DRAWINGS

Referring to the drawings:

FIG. 1 is a perspective view of a feeding syringe assembly for an endoscopic gastronomy tube, the feeding syringe assembly constructed and arranged in accordance with the principle of the invention;

FIG. 2 is fragmented, side elevation view of the assembly of FIG. 1, in which a portion of a stand of the assembly is shown in vertical cross section illustrating the attachment of a syringe support to the stand;

FIG. 3 is a top plan view of a syringe support attached to a stand of the assembly of FIG. 1; and

FIG. 4 is a perspective view of the assembly of FIG. 1 shown as it would appear in use.

### DETAILED DESCRIPTION

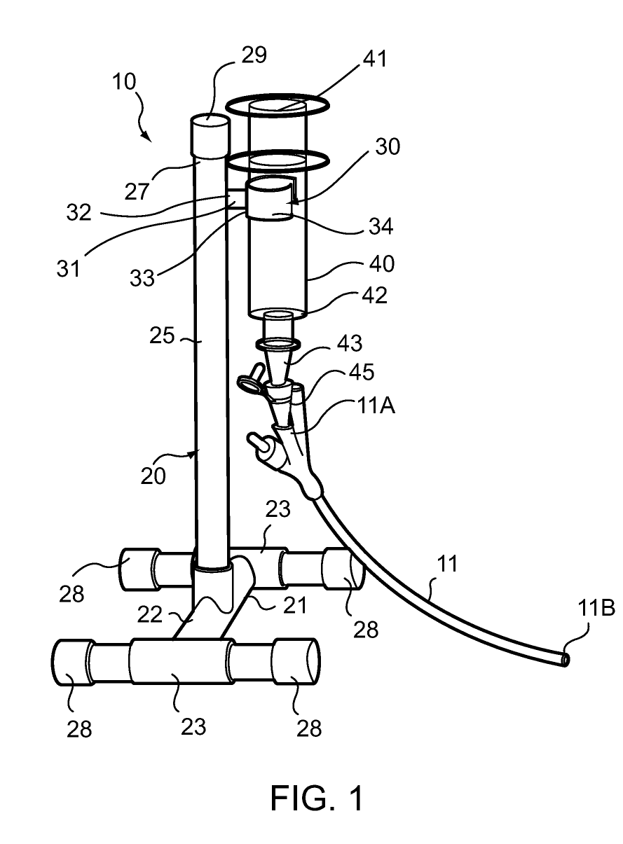

Turning now to the drawings, in which like reference characters indicate corresponding elements throughout the several views, attention is first directed to FIG. 1, in which there is seen a feeding syringe assembly 10 for a percutaneous endoscopic gastronomy (PEG) tube 11. Assembly 10 has a support stand 20, which includes a base 21 and a riser 25, a syringe 40, and a syringe support 30 coupling syringe 40 to support stand 20 to support syringe at an elevated location to receive and gravity convey a formulated food solution to tube 11. Base 21 is provided to be set against a support surface to support stand 20 in place, and consists of a central, elongate support 22 having a stabilizing leg 23 secured at either end thereof. Legs 23 formed at the respective ends of elongate support 22 oppose one another, are substantially equal in size and shape, and are substantially parallel with respect to each other. Extending substantially vertically upright from elongate support 22 between opposed legs 23 is riser 25. Riser 25 is elongate and has a lower end 26 affixed to elongate support 22 between legs 23, and extends substantially vertically upright from lower end 26 to an opposed upper end 27.

In the present embodiment, support stand 20 is formed of elongate, hollow, tubing or stock, formed of polyvinyl chloride or other similarly substantially rigid material or combination of materials, interconnected by way of adhesive, fittings, welding, or the like. Stand 20 is lightweight and easy to pick up, such as by hand, and the ends of legs 23 are formed with non-marking, non-slip feet 28 to keep stand 20 from sliding around when base 21 is set against a support surface. Upper end 27 of riser 25 is formed with a cap 29, which seals upper end 27.

Riser 25 of stand 20 has a syringe support 30. Syringe support 30 is formed proximate to upper end 27, and projects outward with respect to riser 25. Syringe support 30 can be fashioned of plastic, but is preferably fashioned of stainless steel, and includes an elongate arm or stem 31 having an inner end 32 affixed to riser 25 adjacent to upper end 26, and projects outward therefrom to an opposed outer end 33 formed with an attached clamp 34. Clamp 34, as seen in FIGS. 1 and 3, is a C-clamp dimensioned and configured to tightly and releasably grasp syringe 40 as in FIG. 1.

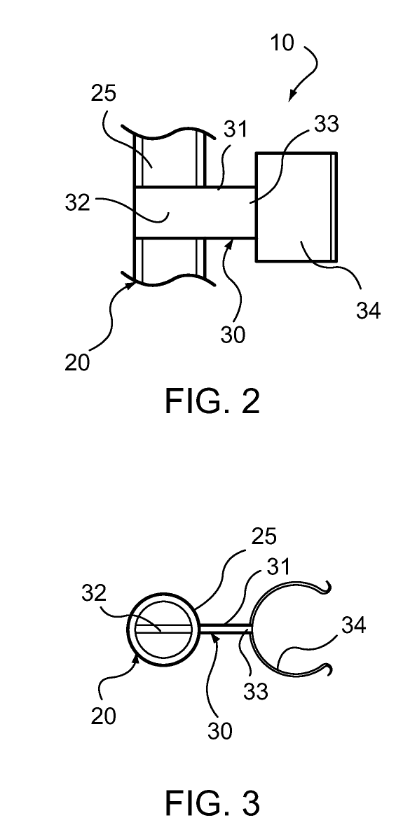

Referencing FIGS. 2 and 3, inner end 32 of stem 31 extends into the interior of riser 25, and is adhesively bonded to secure it in place. Inner end 32 of stem 31 can be affixed to riser 25 in other ways, such as with one or more screws, rivets, clamps, or the like. Clamp 34 receives and clamps syringe 40 as shown in FIG. 1, and grips syringe 40 tightly during use so syringe 40 is prevented from slipping down. The stainless steel from which clamp 34 is made has a spring or spring-like character, which allows a user to easily press syringe 40 into place in clamp 34, and then remove syringe 40 from clamp 34.

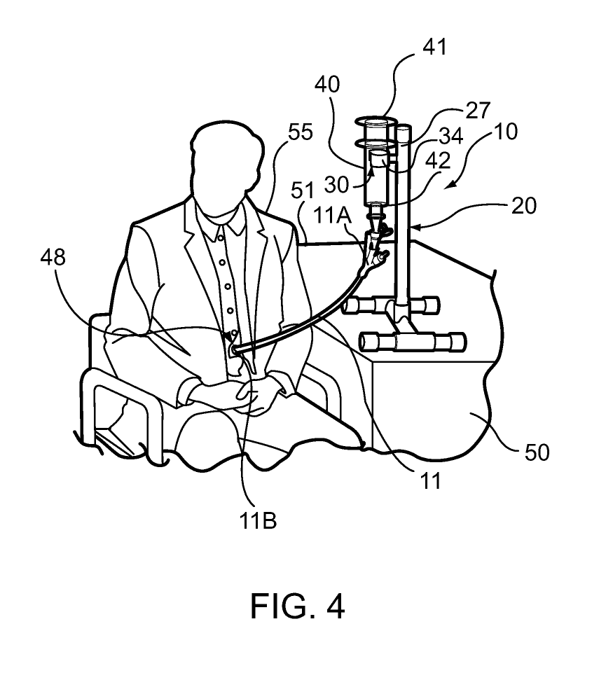

Looking to FIG. 1, syringe 40 is a graduated cylinder and has an open upper end 41 through which syringe 40 is loaded with a formulated food solution. A tapered nipple 43 is formed in lower end 42 of syringe 40, which coupled in fluid communication to inlet 11A of tube 11 with a fitting. Tube 11 has an outlet 11B opposing inlet 11A, which, as shown in FIG. 4, is inserted into the stomach (not shown) of a patient 55 through a tube insertion point or simply an insertion point, denoted generally at 48, in order to convey a formulated food solution to the stomach of patient 55 from syringe 40 via tube 11. Tube 11 couples the stomach of patient 55 in fluid communication with syringe 40 to convey a formulated food solution from syringe 40 to the stomach of patient 40. Accordingly, tube 11 couples patient 55 in fluid communication with syringe 40 to convey a formulated food solution to patient 55 to provide patient 55 with nutrition.

In use, base 21 of stand 20 is placed on a support surface, such as top 50 of desk 51 in FIG. 4 or other support surface, such that riser 25 project substantially vertically upright with respect to base 21 and top 50 of desk to syringe support 30 formed proximate to upper end 27 of riser 25 thereby locating and supporting syringe support 30 at an elevated location. With syringe support 30 supported at an elevated location, syringe 40 held by syringe support 30 as shown in FIG. 4 is held substantially vertically upright at an elevated location well above insertion point 48 for outlet 11B of tube 11 in patient 55, who is shown seated next to assembly 10 in the present example. Outlet 11B of tube 11 is fitted by a physician directly into the stomach of patient 55 through insertion point 48 in patient 55 to allow feeding with assembly 10. When a formulated food solution, such as the formulated food solution offered under the trademark Ensure® or other selected formulated food solution, is placed within syringe 40 through open upper end 41 of syringe 40, gravity forces the formulated food solution into inlet 11A of tube 11 from nipple 43 formed in lower end 42 of syringe 40, and through tube 11 and into the stomach of the patient through outlet 11A extending through insertion point 48 formed in patient 48 thereby eliminating the need for a specialized pump or the assistance of a nurse or caregiver. After use, syringe 40 is removed for washing in preparation for storage and the next feeding period.

The present invention is described above with reference to a preferred embodiment. However, those skilled in the art will recognize that changes and modifications may be made in the described embodiment without departing from the nature and scope of the present invention. Various changes and modifications to the embodiment herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A feeding syringe assembly, comprising:
a tube coupling a patient in fluid communication with a syringe to convey a formulated food solution to a patient to provide the patient with nutrition, the tube connected to the patient at an insertion point;
a stand;
a syringe support coupling the syringe to the stand holding the syringe at an elevated location with respect to the insertion point of the tube to allow a formulated food solution to gravity convey through the tube to the patient from the syringe.

2. The feeding syringe assembly according to claim 1, wherein the syringe support includes a clamp connected to the stand releasably gripping the syringe.

3. The feeding syringe assembly according to claim 2, wherein the clamp is a C-clamp.

4. A feeding syringe assembly, comprising:
a tube coupling a patient in fluid communication with a syringe to convey a formulated food solution to a patient to provide the patient with nutrition, the tube connected to the patient at an insertion point;
a stand including base and a riser extending substantially vertically upright therefrom to an upper end;
a syringe support attached to the riser proximate to the upper end, the syringe support coupling the syringe to the riser holding the syringe at an elevated location with respect to the insertion point of the tube to allow a formulated food solution to gravity convey through the tube to the patient from the syringe.

5. The feeding syringe assembly according to claim 4, wherein the syringe support includes a clamp connected to the stand releasably gripping the syringe.

6. The feeding syringe assembly according to claim 5, wherein the clamp is a C-clamp.
